(19) United States
(12) Patent Application Publication
(10) Pub. No.: US 2025/0035519 A1
(43) Pub. Date: Jan. 30, 2025

(54) DEVICE FOR PURIFYING ADIPOSE TISSUE

(71) Applicant: NEOSYAD, AIX-EN-PROVENCE (FR)

(72) Inventors: Régis ROCHE, LORGUES (FR); François CABAUD, EGUILLES (FR); Xavier NELISSEN, VILLERS SAINT SIMEON (BE)

(21) Appl. No.: 18/717,107

(22) PCT Filed: Oct. 21, 2022
(86) PCT No.: PCT/FR2022/052006

§ 371 Date: Jun. 6, 2024

(30) Foreign Application Priority Data
Dec. 8, 2021 (FR) 2113135

(51) Int. Cl. G01N 1/28

(52) U.S. Cl. CPC G01N 1/286; G01N 2001/2866

(57) ABSTRACT

The present application relates to a device for purifying adipose tissue, comprising at least one sealed enclosure, a filter present in the sealed enclosure, said filter delimiting a centrifugation chamber for centrifuging adipose tissue, and a means for rotating the filter, the filter having a pore size configured to allow a liquid medium to pass and to retain adipose tissue.

### CROSS REFERENCE TO RELATED APPLICATIONS

This application claims priority to PCT Application No. PCT/FR2022/052006 filed on Oct. 21, 2022 which claims priority to FR2113135 filed on Dec. 8, 2021, the contents of each of which are included by reference herein.

### TECHNICAL FIELD

The present application relates to a device for purifying adipose tissue. The application has application, in particular, in “lipofilling” for aesthetic or reconstructive purposes. The application can be used, in particular, in lipofilling useful for mammary surgery operations, the use of the device according to the application not however being limited to this application.

### PRIOR ART

Lipofilling operations or autologous grafting of adipose tissue are used in surgery, especially in surgery on the buttocks to increase volume or in mammary surgery in order to model the breast in order to give it a more natural appearance after a Deep Inferior Epigastric Perforator (DIEP), a reconstruction using a latissimus dorsi flap, or after the insertion of a mammary implant. These operations consist of taking fat from a donor region of a patient, in order to then reintroduce it into the region of interest in the patient's body.

However, the known fat transplant techniques have an important disadvantage, namely a high rate of resorption of the implanted fat. More specifically, a resorption of at least 50% of the implanted fat (loss of volume) is generally observed six months after the transplant operation. Consequently, it is often necessary to operate on the patient several times in order to obtain a final satisfactory result. These repeated operations are undesirable in terms of patient comfort, as well as the cost and duration of treatment.

The adipose tissue collected contains a liquid medium mainly consisting, on the one hand, of oil and blood initially present in the adipose tissue and, on the other hand, a liquid such as a physiological solution introduced during the taking of the adipose tissue and/or during a washing operation thereof.

The inventors have determined that the rate of resorption is mostly linked to the quantity of liquid present in the adipose tissue reintroduced into the body of the patient. In other words, the more liquid which remains in the reinjected adipose tissue, the higher the rate of resorption.

It is therefore important to treat the adipose tissue before its reintroduction into the body of the patient, in order to remove as much of the liquid medium as possible. Document US 2020/0054824 discloses a lipofilling system which uses a gravity filtration device marketed under the tradename Revolve®.

However, this type of filtration device does not allow a sufficient quantity of liquid to be removed from the adipose tissue so as to be able to significantly reduce the rate of resorption.

Another solution consists of centrifuging the adipose tissue in order to separate the phases by density. This solution also does not allow an optimum quantity of liquid to be removed from the adipose tissue. In addition, for the separation of phases to be effective, the centrifugation must be performed at high centrifugal accelerations, generally greater than or equal to 400 g, and very often even reaching 900 g. These speeds/accelerations are harmful for the tissue, and cause cell death typically at a level of between 10% and 40%, depending on the accelerations produced.

### DISCLOSURE

The present application aims to overcome the disadvantages of the prior art by proposing a device for purifying adipose tissue, comprising at least one sealed enclosure, a filter present in the sealed enclosure, said filter delimiting a centrifugation chamber for centrifuging adipose tissue, and a means for rotating the filter, the filer having a pore size configured to allow a liquid medium to pass and to retain adipose tissue.

The purification device of the application thus advantageously combines the use of a filter that is able to drain a liquid medium from the adipose tissue, with setting it in rotation. More specifically, since the filter forms a centrifugation chamber, adipose tissue present inside this chamber can be subjected to centrifugal accelerations against the inner wall of the filter. The liquid medium present in and around the adipose tissue is then effectively drained to the outside of the centrifugation chamber, through the pores of the filter, while the adipose tissue is retained in the chamber. Thus, a large quantity of liquid is removed, allowing purified adipose tissue to be obtained which, once re-implanted, has a resorption rate much lower than that obtained with filtration solutions of the prior art.

The purification device of the application also has the advantage of being able to drain a significant quantity of liquid from the adipose tissue, and to do this without damaging it. Indeed, the solutions of the prior art which use centrifugation in order to separate the phases by density and remove the liquid are implemented in closed enclosures such as tubes or syringes. In order for the phase separation to be effective, the centrifugation must be performed at high centrifugal accelerations, generally greater than or equal to 400 g, and very often even reaching 900 g. These speeds/accelerations are harmful for the adipose tissue, and cause cell death at a level of typically between 10 and 40%, depending on the accelerations produced. In the purification device of the application, the centrifugation chamber is not closed since its wall is formed by a filter. The evacuation of liquid is no longer achieved by phase separation, but by passage through the pores of the filter. It is therefore not necessary to rotate the device rapidly in order to evacuate the liquids. An acceleration that is generally between 5 g and 25 g is sufficient. As a result, the adipose tissue undergoes very little damage. Furthermore, the oil that is potentially generated (a marker of cell death and the reinjection of which must be avoided because it can form oily cysts) is advantageously removed, also due to the filter, which is not the case during centrifugation by phase separation, where the oil is located above the tissue.

The purification device further comprises a collector plate present in the centrifugation chamber, the collector plate being able to move in translation along the axis of rotation of the filter. The collector plate acts as a piston which can scrape the inner wall of the filter so as to collect a maximum of adipose tissue and to facilitate the collection of the purified adipose tissue at the top of the device.

According to a particular aspect, the purification device further comprises a threaded rod extending in the centrifugation chamber and cooperating with a tapped portion of the collector plate, the threaded rod being connected to rotary drive means of the filter, said means being configured to drive the threaded rod in rotation, in a direction of rotation opposite to the direction of rotation of the filter. Here, the same rotary drive means is advantageously used both for the centrifugation of the filter and for the movement in vertical translation of the collector plate.

According to another particular aspect, the purification device further comprises a protective sleeve surrounding the threaded rod. This sleeve can prevent the threaded rod from coming into contact with the adipose tissue which by accumulating at the points where the plate is tapped, can block the movement thereof.

In an embodiment, the purification device further comprises a stiffening element present between the sealed enclosure and the filter.

In another exemplary embodiment, the filter is made of a rigid self-supporting material. In this case, a stiffening element is not necessary in the purification device. The rotary drive means of the filter can be motorised, for example with an electric motor, or can be manual.

### BRIEF DESCRIPTION OF THE FIGURES

FIG. 1 is an exploded schematic view of a purification device in accordance with an exemplary body of the application,

FIG. 2 is a schematic sectional view of the purification device of FIG. 1, once assembled,

FIG. 3 is a schematic sectional view showing the purification device of FIG. 2 after movement of the collector plate.

### DESCRIPTION OF THE EMBODIMENTS

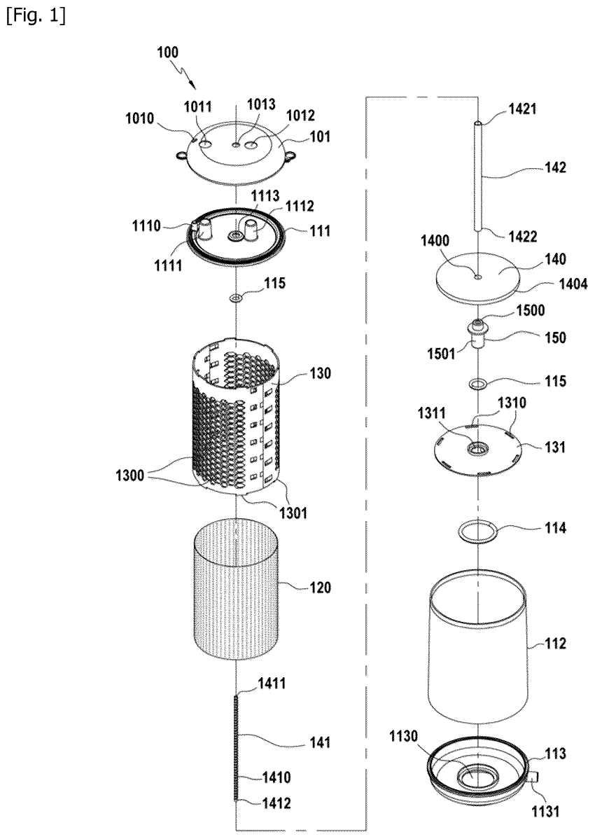

FIGS. 1 to 3 illustrate a purification device 100 according to an embodiment of the application.

The purification device 100 comprises a sealed enclosure 110, formed here by a cap 111, a cylindrical wall 112 and a bottom 113. These elements are fixed together in a sealed manner.

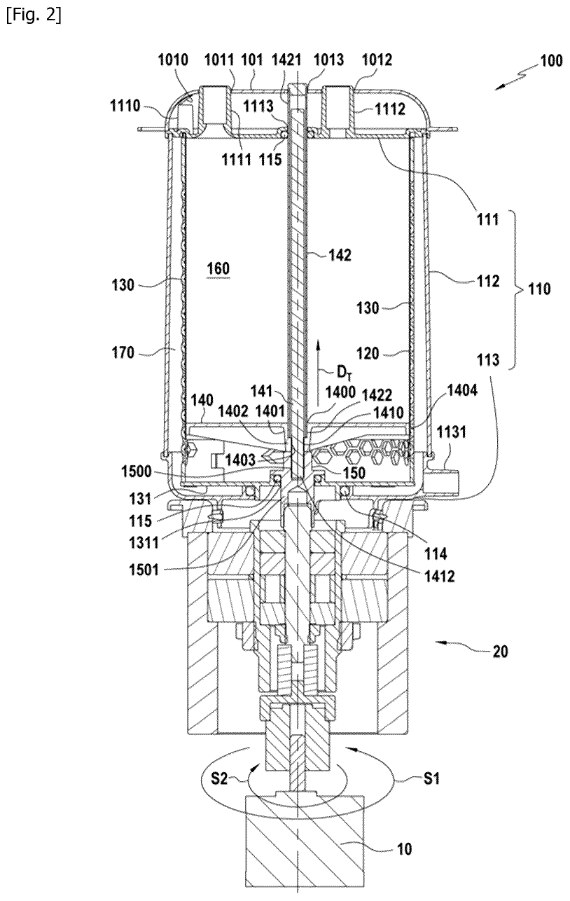

The purification device 100 also comprises a filter or screen 120 present in the sealed enclosure 110. The filter 120 delimits a centrifugation chamber 160 (FIG. 2), the operation of which is described below. In the example described here, the filter 120 has a cylindrical shape. The filter can have other shapes suitable for centrifugation. The filter 120 has a pore size that is configured to allow a liquid medium to pass and to retain adipose tissue. The pore size is chosen, in particular, to enable the passage of liquids such as oil, blood, water or a physiological solution, while retaining the adipose tissue. By way of non-limiting examples, the pore size of the filter can be between 50 μm and 1500 μm, and more preferably between 200 μm and 500 μm. The filter can be produced in different ways, for example by braiding, welding plastic threads or from a material comprising apertures or holes having dimensions corresponding to the desired size. The filter can, in particular, be made of a polymer material such as polyester or polypropylene but a person skilled in the art will know that other materials can be used.

In the example described here, a cylindrical stiffening element 130 is present between the cylindrical wall 112 of the sealed enclosure 110 and the filter 120. Its function, in particular, is to ensure the structural stability of the filter 120 during centrifugation. The stiffening element 130 is made, for example, from metal or plastic material and has a perforated structure defining a plurality of openings 1300 in order to enable the evacuation of the liquid medium drained by the filter 120. The stiffening element 130 is associated with a rotary plate 131. More precisely, at its lower end, the stiffening element 130 has teeth 1301 which cooperate with grooves 1310 that are present in the vicinity of the outer periphery of the rotary plate 131. Of course, any means of securing the stiffening element with the rotary plate, such as clipping or gluing, for example is possible. The rotary plate 131 is connected to a rotary drive means which can be manual or motorised. In the example described here, the rotary plate is connected to an electric motor 10 via a bidirectional clutch 20 configured to ensure the driven rotation of the rotary plate 131 in a first direction of rotation S₁ of the electric motor 10 (FIG. 2). The electric motor 10 can be, for example, a stepper motor or a brushless DC motor. The bidirectional clutch can be replaced by any device enabling a selective driven rotation in two opposite directions of rotation. Seals 114 and 115 are respectively placed below and above the rotary plate 131 in order to ensure the sealing in the lower part of the purification device.

The centrifugation is performed by rotating the filter 120. More precisely, the electric motor 10 is controlled in a first direction of rotation S₁ in order to drive the rotary plate 131 and the stiffening element 130 into engagement with the plate 131. The rotation of the plate 131 and of the stiffening element 130 drives the rotation of the filter 120. The filter is fixed to the stiffening element by any type of suitable means. By way of non-limiting examples, the filter can be fixed using one or more of the following means: glueing, engagement in wedging notches present on the stiffening element, clipping. The speed of the electric motor 10 is controlled so as to apply a centrifugal acceleration to the adipose tissue present in the centrifugation chamber. Thus, adipose tissue present in the centrifugation chamber 160 will be subjected to a centrifugal force against the inner wall of the filter 120, which makes it possible to effectively drain the liquid medium present in the adipose tissue without damaging this.

The centrifugation implemented can be produced by imposing on the filter 120 acceleration greater than or equal to 8 g, for example greater than or equal to 10 g or greater than or equal to 12 g during all or part of the centrifugation. This acceleration, measured in g, corresponds to the ratio between the acceleration undergone by the material and the acceleration due to the Earth's gravity, which is approximately 9.81 m²/s. The acceleration undergone by the material corresponds to the ratio of the centrifugal force applied and the mass of the material considered. The applied centrifugal force is equal to m*ω²*R where m is the mass of the object considered, w is the angular velocity of the filter 120 expressed in rad/s and R is the distance from the axis of rotation X at the centre of gravity of the object considered.

During a centrifugation phase, the adipose tissue and the contaminant materials can undergo an acceleration less than or equal to 40 g, for example less than or equal to 30 g, or even less than or equal to 25 g or less than or equal to 20 g. This acceleration can be between 8 g and 40 g or between 8 g and 30 g or between 8 g and 25 g or between 8 g and 20 g. This acceleration can be between 10 g and 40 g or between 10 g and 30 g or between 10 g and 25 g or between 10 g and 20 g. This acceleration can be between 12 g and 40 g or between 12 g and 30 g or between 12 g and 25 g or between 12 g and 20 g. The values of acceleration described above are sufficient to allow a very significant removal of liquids present in the adipose tissue and, in particular, the interstitial water and to yet further improve the quality of the purified adipose tissue. This last point is more difficult to realise in the case of filtration by gravity, as described in US 2020/0054824. Furthermore, the fact of limiting the acceleration to values well below those used in conventional centrifugation as described above, also participates in obtaining an optimum quality adipose tissue with a low quantity of residual interstitial water enabling it to be easily injected into the patient's body.

The centrifugation can have a duration greater than or equal to 10 seconds, for example between 10 seconds and 60 seconds, preferably between 15 and 45 seconds.

During the centrifugation, the liquid medium passing through the filter 120 and the stiffening element 130, is collected in a volume 170 delimited between the stiffness element 130 and the cylindrical wall 112 of the enclosure 110. The liquid is then removed via an evacuation port 1131 present on the bottom 113 of the enclosure 110.

In the example described here, the cap 111 comprises three ports 1110, 1111 and 1112 intended to be connected, respectively, to a suction device for adipose tissue collected from the body of a patient, to a device for delivering washing liquid, for example a physiological solution, and to a device for re-implantation or reintroduction of adipose tissue. The purification device comprises a cover 101 having openings 1010, 1011 and 1012 which cooperate with the ports 1110, 1111 and 1112 of the cap 111.

Optionally, the purification device can further comprise a collector plate that can move inside the centrifugation chamber. As illustrated in the examples of FIGS. 1 and 2, the purification device 100 further comprises a collector plate 140 which can move in translation in a direction Dr along the axis X of the filter 120. More precisely, the purification device 100 comprises a threaded rod 141 which extends vertically inside the centrifugation chamber 160 following the axis X of the filter 120. The lower end 1412 of the threaded rod is connected to the electric motor 10 by means of a guide 150 and the bidirectional clutch 20. The guide 150 comprises a housing 1500 in which the lower end 1412 is fixed. The lower portion 1501 of the guide 150 is connected to a portion of the bidirectional clutch 20 which is engaged with the electric motor 10 when this transmits a rotary movement in a second direction of rotation S₂ opposite to the first direction of rotation S₁ used for the centrifugation. When the electric motor 10 turns in the first direction of rotation S₁, no rotary movement is transmitted by the bidirectional clutch 20 to the guide 150 to which the threaded rod 141 is connected. Similarly, when the motor 10 turns in the second direction of rotation S₂, no rotary movement is transmitted by the bidirectional clutch 20 to the rotary plate 131 and, consequently, to the filter 120. On its upper face, the collector plate 140 has a central opening 1400 prolonged by a neck 1401 which extends from the inner face of the plate 140. The neck 1401 comprises a portion 1402 comprising a tapping 1403 which cooperates with a thread 1411 of the threaded rod 141. Thus, when the threaded rod is rotated in the second direction of rotation S₂, the collector plate 140 rises in the centrifugation chamber 160 in the direction DT.

The peripheral edge 1404 of the collector plate 140 is opposite the inner wall of the filter 120. Thus, when the collector plate 140 is moved in vertical translation in direction DT, it acts as a piston which can scrape the inner wall of the filter so as to collect as much adipose tissue as possible. The vertical translation of the plate 140 also makes it possible to place the adipose tissue collected as close as possible to the cap 111, and consequently to facilitate its collection for reimplantation.

According to a particular aspect, the threaded rod 141 can be housed in a protective sleeve 142. The protective sleeve 142 which extends between an upper end 1421 and a lower end 1422 can prevent the threaded rod 141 from coming into contact with the adipose tissue which by accumulating at the tapping 1403 in the collector plate 140 can block the movement thereof.

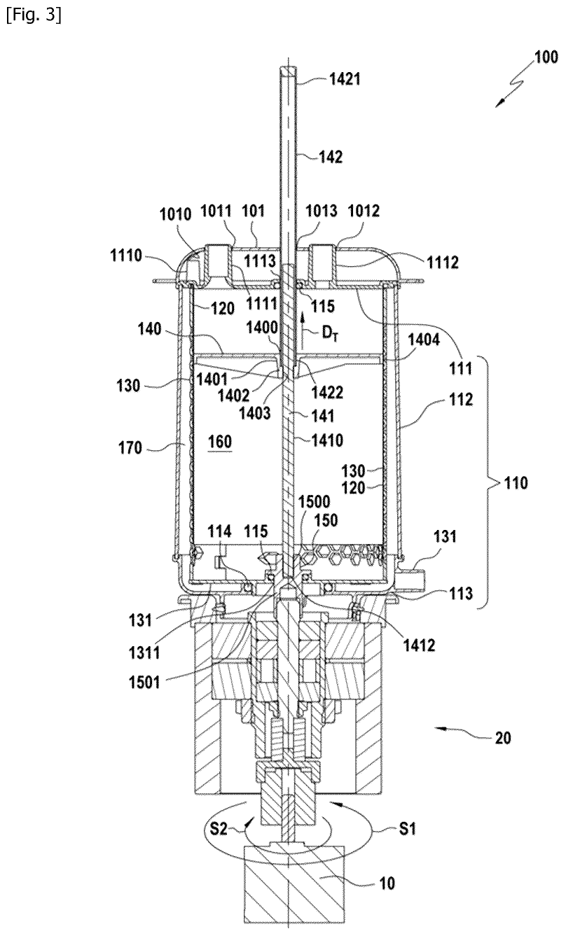

FIG. 3 shows the purification device 100 after operation of the electric motor 10 in the second direction of rotation S₂ enabling the driven rotation of the threaded rod 141. The rotation of the threaded rod 141 drives the vertical translation of the collector plate 140 in the direction DT.

The lower end 1422 of the protective sleeve is fixed in the opening 1400 of the collector plate 140. In order to enable the movement of the protective sleeve 142 during movement of the plate 140, the cap 111 and the cover 101 respectively comprise an opening 1113 and an opening 1013, through which the sleeve 142 slides. A seal 115 is present around the opening 1113 in order to maintain the sealing at the top of the centrifugation chamber.

It is noted that when the purification device does not comprise a collector plate that can move in translation, a bidirectional clutch is not necessary and the electric motor or any other rotary drive means can be directly connected to the rotary plate. In another exemplary embodiment, the filter is made of a rigid self-supporting material. The filter can be produced, in particular, from a metal strip in which holes are produced by laser, water jet or chemical cutting in order to form a screen, a metal grid woven with wires, or beads agglomerated by sintering of metal or ceramic powders. In this case, the stiffening element 130 is no longer necessary and the self-supporting filter is directly engaged with the rotary plate 131.

1. A device for purifying adipose tissue, comprising at least one sealed enclosure, a filter present in the sealed enclosure, said filter delimiting a centrifugation chamber for centrifuging adipose tissue, and a means for rotating the filter, the filter having a pore size configured to allow a liquid medium to pass and to retain adipose tissue, wherein the device further comprises a collector plate present in the centrifugation chamber, the collector plate being able to move in translation along the axis of rotation of the filter.

2. The device according to claim 1, further comprising a threaded rod extending in the centrifugation chamber and cooperating with a tapped portion of the collector plate, the threaded rod being connected to rotary drive means of the filter, said means being configured to drive the threaded rod in rotation, in a direction of rotation opposite to the direction of rotation of the filter.

3. The device according to claim 2, further comprising a protective sleeve surrounding the threaded rod.

4. The device according to claim 1, further comprising a stiffening element present between the sealed enclosure and the filter.

5. The device according to claim 1 to 3, wherein the filter is made of a rigid self-supporting material.

6. The device according to claim 1, wherein the rotary drive means of the filter is manual.

7. The device according to claim 1, wherein the rotary drive means of the filter comprises an electric motor.

8. A device for purifying adipose tissue, comprising:
at least one sealed enclosure;
a filter present in the sealed enclosure, said filter delimiting a centrifugation chamber for centrifuging adipose tissue, wherein the filter has a pore size configured to allow a liquid medium to pass and to retain adipose tissue; and
a collector plate present in the centrifugation chamber, the collector plate being able to move in translation along an axis of rotation of the filter.

9. The device according to claim 8, further comprising a threaded rod extending in the centrifugation chamber and cooperating with a tapped portion of the collector plate, the threaded rod being connected to rotary drive means of the filter, said means being configured to drive the threaded rod in rotation, in a direction of rotation opposite to the direction of rotation of the filter.

10. The device according to claim 9, further comprising a protective sleeve surrounding the threaded rod.

11. The device according to claim 8, further comprising a stiffening element present between the sealed enclosure and the filter.

12. The device according to claim 8, wherein the filter is made of a rigid self-supporting material.

13. The device according to claim 8, wherein the filter is driven manually.

14. The device according to claim 8, wherein the filter is driven with an electric motor.
